(19) United States
(12) Patent Application Publication
(10) Pub. No.: US 2007/0239233 A1
(43) Pub. Date: Oct. 11, 2007

(54) Surface mount light emitting diode medical apparatus

(75) Inventor: Gerald Klein, Tamarac, FL (US)

Correspondence Address: THOMAS D. HELMHOLDT;YOUNG & BASILE P C, 3001 WEST BIG BEAVER RD., SUITE 624, TROY, MI, 48084, US

(73) Assignee: LIFE WITHOUT PAIN L.L.C., Fort Lauderdale, FL (US)

(21) Appl. No.: 11/400,153

(22) Filed: Apr. 7, 2006

(51) Int. Cl. A61N 5/06

(52) U.S. Cl. 607/88; 607/90

(57) ABSTRACT

An apparatus for relief of chronic and acute pain in tissue or arthritic joints and/or for promoting wound healing and human tissue growth can include a handheld housing supporting an array of light emitting diodes (LED) for application of light to a portion of a human body. The LED array can have a light output in a wavelength range of between approximately 630 nanometers to approximately 940 nanometers, inclusive, and a power intensity in a range of at least approximately 20 milliwatts per square centimeter. A control interface can be include buttons for controlling operation of the LED array, and a timer operable in response to pressing the buttons for delivering power to the LED array for a predetermined period of time. The control interface can include an over-temperature sensor operable to automatically turn off power delivery to the LED array when a sensed temperature is above a predetermined value.

### FIELD OF THE INVENTION

The present invention relates to surface mounted light emitting diode medical devices for use in chronic and acute pain management in tissue or arthritic joints and/or promoting wound healing and human tissue growth.

### BACKGROUND

Laser light and hyperbaric oxygen have been widely acclaimed to speed wound healing in ischemic, hypoxic wounds. Lasers can provide low energy stimulation of tissue, which can result in increased cellular activity during wound healing. Some of the cellular activities can include increased fibroblast proliferation, growth factor syntheses, collagen production and angiogenesis. Inherent characteristics of lasers can make the use of lasers problematic. These limitations can include limitations of wavelengths and beam widths. The wavelengths of light optimal for wound healing cannot be efficiently produced by lasers, and the size of wounds capable of being treated by a laser is limited.

Light emitting diodes (LED) are an effective alternative to lasers. LEDs can be designed to produce various wavelengths and can be arranged in large, arrays, allowing treatment of large wounds. LEDs can stimulate wound healing at near-infrared wavelengths from 630 to 940 nanometers (nm) in laboratory animals, and have been approved by the U.S. Food and Drug Administration for human usage. Near infrared LED light can increase the growth of fibroblast and muscle cells in tissue culture. LED treatments can have many potential applications, including treatment of serious burns, crash injuries, non-healing fractures, muscle and bone atrophies, traumatic ischemic wounds, radiation tissue damage, compromised skin grafts, and aid in tissue regeneration.

Biologists have found that cells exposed to near-infrared light from LEDs can grow 150% to 200% faster than those cells not stimulated by such light. This form of light can increase energy inside cells, which result in speeding up the healing process. In U.S. Pat. No. 4,930,504 Diamantopoulos describes an LED based “Device for Biostimulation of Tissue and Method for Treatment of Tissue” in which 9 examples are provided of positive treatment results. In the proceedings of the 20^{th} World Congress of Dermatology, Paris, July 2002, Dr B. Deshpanda described the curing of a “non-healing diabetic ulcer” by the use of a 660 nm wavelength red light device. Other researchers have performed clinical studies that indicate that pain relief and healing are accomplished with LED wavelengths from 630 to 940 nanometers.

### SUMMARY

It would be desirable in an embodiment of the present invention to provide a lightweight, user friendly, handheld unit incorporating surface mount light emitting diodes for a medical treatment apparatus that can be used by an individual without professional assistance for alleviation of chronic and acute pain in tissue or arthritic joints and/or for promoting wound healing and human tissue growth. It would be desirable in an embodiment of the present invention to provide a device and method of operation for reducing pain by exposing tissue to LED light whose wavelength is in a region that has been shown to produce pain reduction and wound healing.

An embodiment of the present invention can include a handheld housing which can include two buttons: a start button and a stop button which are integrated into or located under a flexible label to prevent protrusions from the housing. The housing can include an over-temperature indicator light. The housing can support a treatment array of LED lights. The housing can enclose an end-of-treatment audio signal or beeper.

Other applications of the present invention will become apparent to those skilled in the art when the following description of the best mode contemplated for practicing the invention is read in conjunction with the accompanying drawings.

### BRIEF DESCRIPTION OF THE DRAWINGS

The description herein makes reference to the accompanying drawings wherein like reference numerals refer to like parts throughout the several views, and wherein:

FIG. 1 is a plan view of a handheld surface mounted light emitting diode medical apparatus for reduction of chronic and acute pain in tissue or arthritic joints and/or for promoting wound healing and human tissue growth according to an embodiment the present invention;

FIG. 2 is a cross-sectional view of the surface mount light emitting diode array and heat dissipation member according to an embodiment of the present invention; and

FIG. 3 is a simplified block diagram showing an embodiment of the handheld treatment unit, including external alternate power sources, and a support wrap capable of eliminating the need to hold a unit during treatment.

### DETAILED DESCRIPTION

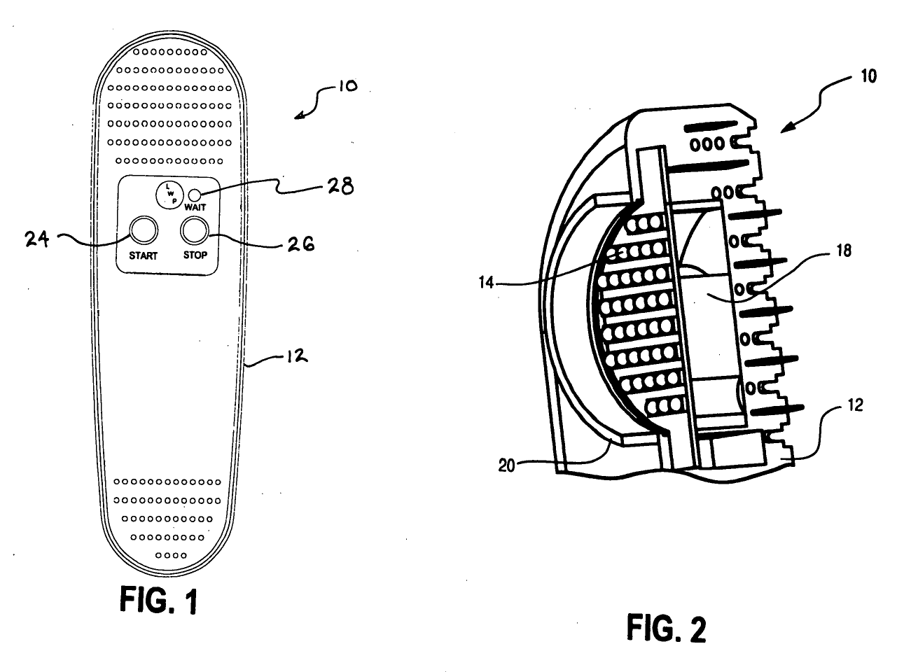

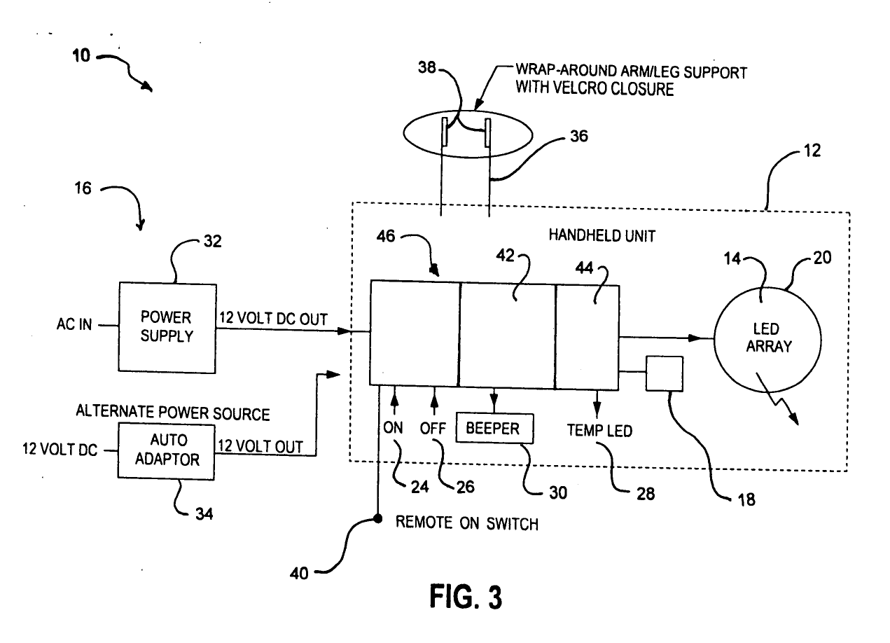

Referring now to FIGS. 1-3, the surface mount light emitting diode medical apparatus 10 according to an embodiment of the present invention can include a handheld portable housing 12. The housing 12 can include at least one button, and preferably two buttons: a start button 24 (FIGS. 1 and 3) and a stop button 26 (FIGS. 1 and 3). The housing 12 can support a delay light (or over temperature indicator light) 28 (FIGS. 1 and 3) and a treatment light array of light emitting diodes (LEDs) 14 (FIGS. 2 and 3). The housing 12 can enclose an audio signal beeper 30 (FIG. 3). The portable, handheld, housing 12 can enclose and support a plurality of surface mount light emitting diodes 14 for reducing chronic and acute pain in tissue or arthritic joints and/or for promoting wound healing and human tissue growth by exposing tissue to light of a specific wavelength, or in a specific range of wavelengths. The housing 12 can contain 81 LEDs which in combination can produce 200 milliwatts of optical energy centered at 630 nanometers wavelength. The surface mount light emitting diodes 14 can be of a single wavelength, or can be constructed to emit light of different wavelengths. A heat dissipation or cooling member 18, such as a heat sink, an electric powered fan or any combination thereof, can be provided to cool the surface mounted light emitting diodes 14. A peripheral ridge 20 can surround the surface mounted light emitting diodes 14 in order to space the LEDs 14 a sufficient distance from the surface of the patient to be treated for best results. The handheld device is designed to operate with 12 volts direct current (dc) input supply 16, supplied either by a wall receptacle plug-in alternating current (ac) powered supply 32 (FIG. 3) operating from 100 to 240 volts at 50 to 60 Hz input, or from an automobile cigarette lighter adapter 34 (FIG. 3). This minimizes the weight of the handheld unit and makes it available for treatment while driving.

In operation, a user can place the apparatus 10 on a portion of the body of a patient to be treated. The user can push the start button 24 (FIG. 3) and the treatment light array 14 can be activated for a desired time interval. By way of example and not limitation, the time interval can be for approximately 240 seconds. At the end of the desired time interval, by way of example and not limitation such as for approximately 4 minutes, the treatment light array 14 can be turned off followed by a short audio signal, such as an end of treatment beep through beeper 30 (FIG. 3). If the user pushes the stop button 26 (FIG. 3) before the predetermined time period has elapsed, the treatment light array 14 can be turned off.

In operation of an embodiment of the present invention, a user can place the treatment device on a portion of the body to be treated. A cloth support band 36 (FIG. 3) with hook and loop fastening end closures 38 (FIG. 3), such as VELCRO™, can be supplied to support the device on the treatment area. The cloth support band 36 (FIG. 3) can eliminate the need for manually holding the housing 12 during treatment, making it safe for use while driving. A plug-in remote start button 40 (FIG. 3) can be provided to allow the user to operate the device when the on-button 24 located on the housing 12 in not within convenient reach of the user. The user can push either start button 24 (FIG. 3) or remote start button 40 (FIG. 3), and the treatment light array 14 can be activated for a predetermined time interval, such as for example approximately 4 minutes. At the end of the predetermined time period, the treatment light array 14 can be turned off, by a timer 42 (FIG. 3), followed by a short end of treatment audible beep through beeper 30 (FIG. 3). If the user pushes the stop button 26 (FIG. 3), before the predetermined time period has elapsed, the treatment light array 14 can be turned off.

An over-temperature sensor 44 (FIG. 3) can be provided to detect if the treatment light array 14 is too hot, and its reliability may be compromised, in which case, the treatment can be terminated and the delay indicator light 28 can be turned on. An internal heat dissipation or cooling member 18, by way of example and not limitation, such as a heat sink, an electric powered fan, or any combination thereof, can continue to operate within the housing 12 until a predetermined lower temperature is achieved. Once a predetermined lower temperature is achieved, the delay indicator light 28 (FIG. 3), and the electric powered fan if present, can be turned off. If the user presses the start button 24 (FIG. 3), or remote start button 40 (FIG. 3), during the over temperature condition, the treatment cycle will not be started and the start button 24 (FIG. 3), or remote start button 40 (FIG. 3) will be ignored until the over temperature condition subsides.

A kit according to an embodiment of the present invention can include an apparatus 10 for the relief of chronic and acute pain management in tissue or arthritic joints and/or for promoting wound healing and human tissue growth. The apparatus 10 can include a housing 12 of sufficiently small size to be handheld, and an array of light emitting diodes (LED) 14 supported by the housing 12 for application of light to a portion of a human body. The LED array 14 can have a light output in a wavelength range of between approximately 630 nanometers to approximately 940 nanometers, inclusive, and a power intensity in a range of at least approximately 20 milliwatts per square centimeter. A heat dissipation or cooling member 18 can be supported by the housing 12 for removing heat from the LED array 14. A user-friendly control interface 46 can be supported by the housing 12 and can have at least one button 24,26 integrated into a flexible label affixed to an external surface of the handheld housing 12 for controlling operation of the LED array 14 during application of light therapy treatment to the portion of the human body to be treated. The control interface 46 can include a timer 42 operable in response to pressing the at least one button 24,26,40 for controlling delivery of power to the LED array 14 for a predetermined period of time. The control interface 46 can include an over-temperature sensor 44 operable to automatically turn off power delivery to the LED array 14 when a sensed temperature is above a predetermined value. The kit can also include at least one 12 volt power supply device 16 selected from a group consisting of a plug-in alternating current (ac) powered supply 32, an automotive cigarette lighter adapter 34, and any combination thereof. A remote start button 40 can be operably connectible with the control interface 46 for starting a light therapy treatment with a button locatable remote from the housing 12. At least one strap 36 or band can be associated with the housing 12 for supporting the housing 12 with respect to the portion of the human body to be treated. A beeper 30 supported by the housing 12 can audibly signal an end of a light therapy treatment to the portion of the human body to be treated.

While the invention has been described in connection with what is presently considered to be the most practical and preferred embodiment, it is to be understood that the invention is not to be limited to the disclosed embodiments but, on the contrary, is intended to cover various modifications and equivalent arrangements included within the spirit and scope of the appended claims, which scope is to be accorded the broadest interpretation so as to encompass all such modifications and equivalent structures as is permitted under the law.

What is claimed is:

1. An apparatus for relief of chronic and acute pain in tissue or arthritic joints and/or for promoting wound healing and human tissue growth comprising:
a housing of sufficiently small size to be handheld;
an array of light emitting diodes (LED) supported by the housing for application of light to a portion of a human body; and
a user-friendly control interface supported by the housing for controlling operation of the array of light emitting diodes during application of light therapy treatment to the portion of the human body to be treated.

2. The apparatus of claim 1 further comprising:
a light output of the array of light emitting diodes in a wavelength range of between approximately 630 nanometers to approximately 940 nanometers, inclusive.

3. The apparatus of claim 1 further comprising:
a power intensity of the array of light emitting diodes in a range of at least approximately 20 milliwatts per square centimeter.

4. The apparatus of claim 1 further comprising:
a direct current (dc) power input connectible to the housing for powering the array of light emitting diodes and the control interface.

5. The apparatus of claim 4 further comprising:
the direct current power input selected from a group of 12 volt power supply devices consisting of a plug-in alternating current (ac) powered supply, an automotive cigarette lighter adapter, and any combination thereof.

6. The apparatus of claim 1 further comprising:
the control interface including a start button and a stop button integrated into a flexible label affixed to an external surface of the handheld housing.

7. The apparatus of claim 1 further comprising:
the control interface including a timer operable in response to pressing a start button for delivering power to the array of light emitting diodes for a predetermined period of time.

8. The apparatus of claim 7 further comprising:
the predetermined period of time for the timer set at approximately 4 minutes.

9. The apparatus of claim 1 further comprising:
a beeper for audibly signalling an end of a light therapy treatment to the portion of the human body to be treated.

10. The apparatus of claim 1 further comprising:
at least one strap connectible to the housing for supporting the housing with respect to the portion of the human body to be treated.

11. The apparatus of claim 10 further comprising:
hook and loop fasteners connected to opposite ends of the at least one strap for engagement with one another to hold the housing in a desired location with respect to the portion of the human body to be treated.

12. The apparatus of claim 1 further comprising:
a heat dissipation member supported by the housing for removing heat from the housing.

13. The apparatus of claim 12 further comprising:
the heat disappation member selected from a group consisting of a heat sink, an electric powered fan, and any combination thereof.

14. The apparatus of claim 12 further comprising:
the control interface including an over-temperature sensor operable to automatically turn off power delivery to the array of light emitting diodes when a sensed temperature is above a predetermined value.

15. The apparatus of claim 14 further comprising:
an over-temperature indictor light supported by the housing for visually indicating an over-temperature condition, the over-temperature indicator light illuminated by the control interface in response to the sensed temperature above the predetermined value.

16. The apparatus of claim 1 further comprising:
a remote start button operably connectible with the control interface for starting a light therapy treatment with a button locatable remote from the housing.

17. A kit including an apparatus for relief of chronic and acute pain and/or for promoting wound healing and human tissue growth comprising:
a housing of sufficiently small size to be handheld;
an array of light emitting diodes (LED) supported by the housing for application of light to a portion of a human body, a light output of the array of light emitting diodes in a wavelength range of between approximately 630 nanometers to approximately 940 nanometers, inclusive, a power intensity of the array of light emitting diodes in a range of at least approximately 20 milliwatts per square centimeter;
a heat disappation member supported by the housing for removing heat from the array of light emitting diodes;
a user-friendly control interface support by the housing and having at least one button for controlling operation of the array of light emitting diodes during application of light therapy treatment to the portion of the human body to be treated, the control interface including a timer operable in response to pressing the at least one button for delivering power to the array of light emitting diodes for a predetermined period of time, the control interface including an over-temperature sensor operable to automatically turn off power delivery to the array of light emitting diodes when a sensed temperature is above a predetermined value; and
at least one 12 volt power supply device selected from a group consisting of a plug-in alternating current (ac) powered supply, an automotive cigarette lighter adapter, and any combination thereof.

18. The kit of claim 17 further comprising:
a remote start button operably connectible with the control interface for starting a light therapy treatment with a button locatable remote from the housing.

19. The kit of claim 17 further comprising:
at least one strap connectible to the housing for supporting the housing with respect to the portion of the human body to be treated.

20. The kit of claim 17 further comprising:
a beeper supported by the housing for audibly signalling an end of a light therapy treatment to the portion of the human body to be treated.
